(19) United States
(12) Patent Application Publication
(10) Pub. No.: US 2010/0292341 A1
(43) Pub. Date: Nov. 18, 2010

(54) QUICK DISSOLVE COMPOSITIONS OF MEMANTINE HYDROCHLORIDE

(75) Inventors: Ramakrishnan Sankar, Madukkur (IN); Chidambaram Seshadri Venkateswaran, Tamil Nadu (IN); Praveen Reddy Billa, Tamil Nadu (IN)

Correspondence Address: SEED INTELLECTUAL PROPERTY LAW GROUP PLLC, 701 FIFTH AVE, SUITE 5400, SEATTLE, WA, 98104, US

(73) Assignee: ORCHID CHEMICALS & PHARMACEUTICALS LIMITED, Nungambakkam (IN)

(21) Appl. No.: 12/666,985

(22) PCT Filed: Jun. 26, 2008
(86) PCT No.: PCT/IB08/01670

§ 371 Date: Jun. 30, 2010

(30) Foreign Application Priority Data
Jun. 29, 2007 (IN) 1394/CHE/2007

(51) Int. Cl. A61K 31/13; C07C 211/38; A61P 25/28

(52) U.S. Cl. 514/662; 564/459

(57) ABSTRACT

The present invention relates to quick dissolve pharmaceutical compositions. More particularly the invention relates to quick dissolve pharmaceutical compositions of memantine hydrochloride capable of dissolving in the oral cavity and process for preparing such compositions. The quick dissolve pharmaceutical compositions of memantine hydrochloride contain at least one water-soluble diluent in particular a mono- or disaccharide and at least one disintegrant and optionally other pharmaceutically acceptable excipients.

### FIELD OF THE INVENTION

The present invention relates to quick dissolve compositions of Memantine hydrochloride capable of dissolving in the oral cavity and processes for preparing such compositions.

### BACKGROUND OF THE INVENTION

Memantine hydrochloride is an adamantine derivative with the following structural formula.

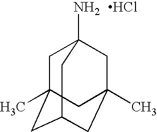

Memantine hydrochloride, designated as 1-amino-3,5-dimethyladamantane hydrochloride is an orally active NMDA receptor antagonist indicated for the treatment of moderate to severe dementia of Alzheimer's disease.

Alzheimer's disease in an illness that involves the loss of neurons in areas of the brain responsible for cognitive functions including language, behavior, learning and memory resulting in clinical manifestations like amnesia, language deterioration and visuospatial deficits. The incidence rate of Alzheimer's disease is estimated to rise from 0.6% per year at 65-69 years to 8.4% per year at age≧85 years, and prevalence increases from 3% in persons aged 65-74: years to 47% in those aged ≧85 years.

Presently memantine hydrochloride is marketed in United States under United Kingdom it is marketed under the proprietary name Exiba in the form of immediate release tablets, oral drops and solution.

However the currently marketed dosage forms present the following disadvantages:
a) As explained above, Alzheimer's disease prevails largely in elderly patients and they have problem in swallowing the conventional tablets dosage forms as these dosage forms need to be swallowed as a whole.
b) In case oral drops and solutions dispensed to contain multiple divided doses, administration from this multiple divided doses may not deliver the required precise dose. Also these dosage forms always require a dosing device to deliver the dose.

Thus there is a need for an alternate unit dosage form of memantine hydrochloride, which will avoid the presented disadvantages. It is an object of the present invention to provide quick dissolve compositions of memantine hydrochloride capable of disintegrating/dissolving in the oral cavity without the need to swallow the composition as a whole.

### SUMMARY OF THE INVENTION

In one general aspect, there is provided a quick dissolve pharmaceutical composition comprising effective amount of memantine hydrochloride, wherein the composition dissolves or disintegrates in less than 60 seconds, preferably in less than 30 seconds.

Embodiments of the composition may include one or more of the following features. For example the composition contains at least one water-soluble diluent and at least one disintegrant and other pharmaceutically acceptable excipients.

The water-soluble diluent may be one or more of mono and di saccharides like mannitol, sorbitol, xylitol, maltitol, sucrose, fructose and lactose, preferably mannitol. The water-soluble diluent present may be from about 0.5% to about 90% by weight of the composition.

The disintegrant may be one or more of crospovidone, croscarmellose sodium, starch, starch derivatives or cellulose polymers, preferably crospovidone. The disintegrant present may be from about 0.5% to about 50% by weight of the composition.

The pharmaceutically acceptable excipients, may be one or more of sweetening agents, flavoring agents, pH modifiers, coloring agents, glidants, lubricants, anti-tacking agents and binders.

In another general aspect of the invention is provided a process for preparing quick dissolve composition comprising a) forming a Mixture of memantine hydrochloride with one or more pharmaceutical excipients b) processing the mixture into an oral dosage form.

In the process, forming of mixture involves wet granulation, dry granulation or direct compression. The mixture is processed into dosage forms like tablet, capsules, pellets or powder.

### DESCRIPTION OF THE INVENTION

This invention relates to a quick dissolve pharmaceutical composition comprising memantine hydrochloride. The “quick dissolve composition” as used herein is interchangeable with fast dissolve, rapid dissolve, rapid melt, quick disintegrating, fast disperse and orally disintegrating compositions and the like. All such dosage forms are adapted to dissolve, disperse or disintegrate rapidly in the oral cavity, resulting in a solution or suspension without the need for the administration of water. Any such composition is consistent with the objects of the invention. The composition of the invention dissolve, disintegrate or disperse in less than 60 seconds preferably in less than 30 seconds.

The composition comprises effective amount of memantine hydrochloride, at least one water-soluble diluent and at least one disintegrant and other pharmaceutically acceptable excipients. An effective amount is the amount or quantity of memantine hydrochloride, which is sufficient to elicit an appreciable biological response when administered to a patient.

The water-soluble diluent in the composition is present in an amount from about 0.5% to about 90% by weight of the composition. The water-soluble diluent may be one or more of mono- and di saccharides like mannitol, sorbitol, xylitol, maltitol, sucrose, fructose and lactose, wherein mannitol is preferred.

The disintegrant in the composition is present in an amount from about 0.5% to about 50% by weight of the composition. The disintegrant may be one or more of cross-linked polyvinyl pyrrolidone (crospovidone), croscarmellose sodium, starch, starch derivatives or cellulose polymers, wherein crospovidone is preferred.

The pharmaceutically acceptable excipients may be one or more of sweetening agents, flavoring agents, pH modifiers, coloring agents, glidants, lubricants, anti-tacking agents and binders.

The sweeteners may be one or more of glucose, dextrose, invert sugar, saccharin and its various salts, acesulfame potasium, dipeptide sweeteners such as neotame, aspartame, sugar alcohols such as sorbitol, mannitol, xylitol and the like.

The flavoring agents may, be chosen, from natural and synthetic flavorants. An illustrative list of such agents include volatile oils, synthetic flavor oils, flavoring aromatic oils, liquids, oleoresins and extracts derived from plants, leaves, flowers, fruits, stems and combinations thereof. A non-limiting representative list for example includes tutti frutti flavor, peppermint flavor, mint flavor, menthol flavor and the like.

Colorants used include food, drug and cosmetic colors (FD&C), drug and cosmetic colors (D&C) or external drug and cosmetic colors. These colors are dyes, lakes, and certain natural and derived colorants. A representative list for example includes iron oxide yellow, iron oxide red, FD&C blue.

The lubricant may be one or more of talc, colloidal silicon dioxide, magnesium stearate, hydrogenated vegetable oil, stearic acid, stearic acid salts and glycols. The glidants include colloidal silicon dioxide, talc and the like. The binders may be one or more of povidone, starch or starch derivatives and cellulose derivatives.

Alternatively the quick dissolve composition of the present invention may be coated with taste mask coating. Useful taste mask coatings can include acrylate/cellulosic polymers. Acrylate polymers may include such as Eudragit RS, Eudragit RL, Eudragit E 100 and Eudragit NE 30 D or mixtures thereof. Cellulosic polymers include ethyl cellulose, hydroxy propyl cellulose (HPC), hyproxy propyl methylcellulose (HPMC) or mixtures thereof.

The quick dissolve composition of the present invention can be formulated into oral dosage forms, in the form of tablets, capsules, pellets, dispersible granules and powder, preferably in the form of tablet.

The pharmaceutical composition of the present invention can be processed into oral dosage forms by conventional process of wet granulation, dry granulation or direct compression. In wet granulation, the drug along with various excipients selected from a diluent, disintegrant and optionally other conventional excipients are mixed, granulated with a suitable granulating fluid followed by drying the damp mass and screening. The dried granules are further processed into desired oral dosage form.

Dry granulation cane be done by blending the drug and excipients selected from a diluent, disintegrant and optionally other conventional excipients followed by slugging/roll compacting, milling, screening, and the resulting granules are processed into desired oral dosage form such as tablets, capsules or pellets.

Alternately the composition can be prepared by direct compression, the process comprising; forming a blend by mixing the drug with a diluent, a disintegrant, and optionally one or more conventional excipients. The above blend can be further processed into the desired oral dosage forms such as tablets, capsules or pellets.

The pharmaceutical compositions of the present invention represent novel oral quick dissolve composition of memantine hydrochloride which composition dissolve, disintegrate or disperse in the oral cavity without the need for the administration of water.

The following examples merely illustrates the invention and do not limit the scope of the invention

### Example 1

| S. No. | Ingredients | Quantity (mg/tab) | % W/W |
|---|---|---|---|
| 1 | Memantine Hydrochloride | 10.000 | 4.96 |
| 2 | Mannitol | 158.350 | 78.58 |
| 3 | Cross linked polyvinyl pyrrolidone | 15.000 | 7.44 |
| | (Crospovidone) | | |
| 4 | Peppermint Flavor | 3.500 | 1.74 |
| 5 | Menthol | 0.500 | 0.25 |
| 6 | Acesulfame Potassium | 6.000 | 2.98 |
| 7 | Aspartame | 1.500 | 0.74 |
| 8 | Iron Oxide Red | 0.075 | 0.04 |
| 9 | Iron Oxide Yellow | 0.075 | 0.04 |
| 10 | Colloidal Silicon dioxide | 2.000 | 0.99 |
| 11 | Magnesium Stearate | 4.506 | 2.23 |

| | | |
|---|---|---|
| Total | 201.506 | 100 |

Memantine Hydrochloride, Mannitol, Acesulfame Potassium, Aspartame, Peppermint flavor, Menthol and Colloidal silicon dioxide were sifted together. Crospovidone, Iron Oxide Red and Iron Oxide Yellow were sifted together separately. Sifted materials were blended in an Octagonal blender. Resulting blend was lubricated with Magnesium stearate. The resulting blend was compressed in to tablets.

### Disintegration:

The tablets were subjected to disintegration test as per United States Pharmacopoeia (USP) and static disintegration time was also evaluated by the following procedure. 20 ml of purified water was taken in a 9 mm petridish. Tablet was placed at the center of the petridish containing purified water. Time taken for complete hydration and disintegration was determined. The results are tabulated below.

TABLE 1

| Test | Disintegration Time (sec) |
|---|---|
| Disintegration Test as per USP | 17 |
| Static Disintegration | 18 |

### Dissolution:

The resulting tablets were also subjected to dissolution test using USP type I dissolution apparatus in 900 ml of dissolution medium (12 g sodium chloride in 6 liter water, pH adjusted to 1.2 with HCl) stirred at 100 rpm. The result is tabulated below.

TABLE 2

| Time (min) | Percentage Drug dissolved |
|---|---|
| 10 | 95.7 |
| 20 | 98.0 |
| 30 | 99.5 |
| 45 | 100.0 |
| 60 | 101.7 |

### Example 2

| S. No. | Ingredients | Quantity (mg/tab) | % W/W |
|---|---|---|---|
| 1 | Memantine Hydrochloride | 10.00 | 4.55 |
| 2 | Mannitol | 158.88 | 72.22 |
| 3 | Cross linked polyvinyl pyrrolidone | 16.50 | 7.50 |
| | (Crospovidone) | | |
| 4 | Peppermint Flavor | 3.90 | 1.77 |
| 5 | Acesulfame Potassium | 6.60 | 3.00 |
| 6 | Aspartame | 3.90 | 1.77 |
| 7 | Iron Oxide Red | 0.25 | 0.11 |
| 8 | Iron Oxide Yellow | 0.17 | 0.08 |
| 9 | Colloidal Silicon dioxide | 8.80 | 4.00 |
| 10 | Sodium Stearyl Fumarate | 11.0 | 5.00 |

| | | |
|---|---|---|
| Total | 220.00 | 100 |

A part of Crospovidone, Iron oxide red and Iron oxide yellow were sifted twice through #80 ASTM sieve. The resulting material was sifted along with Memantine Hydrochloride, a part of Colloidal Silicon dioxide, Mannitol, remaining portion of Crospovidone, Peppermint flavor, Acesulfame Potassium and Aspartame through #40 ASTM sieve. To this Sodium Stearyl Fumarate and remaining portion of Colloidal Silicon dioxide sifted together through #60 ASTM sieve was added and blended in a blender. The resulting blend was sifted through #40 ASTM sieve and after blending for required time compressed into tablets using suitable tooling.

### Disintegration:

The tablets were subjected to disintegration test as per United States Pharmacopoeia (USP) and the result is tabulated below.

TABLE 3

| Test | Disintegration Time (sec) |
|---|---|
| Disintegration Test as per USP | 13 |

### Dissolution:

The resulting tablets were also subjected to dissolution test using USP type dissolution apparatus in 900 ml of dissolution medium (12 g sodium chloride in 6 liter water, pH adjusted to 1.2 with HCl) stirred at 100 rpm. The result is tabulated below.

TABLE 4

| Time (min) | Percentage Drug dissolved |
|---|---|
| 05 | 98.9 |
| 10 | 101.6 |
| 15 | 102.2 |
| 20 | 101.9 |

### Example 3

| S. No. | Ingredients | Quantity (mg/tab) | % W/W |
|---|---|---|---|
| 1 | Memantine Hydrochloride | 10.00 | 4.55 |
| 2 | Mannitol | 44.00 | 20.00 |
| 3 | Lactose Monohydrate | 114.88 | 52.22 |
| 4 | Cross linked polyvinyl pyrrolidone | 16.50 | 7.50 |
| | (Crospovidone) | | |
| 5 | Peppermint Flavor | 3.90 | 1.77 |
| 6 | Acesulfame Potassium | 6.60 | 3.00 |
| 7 | Aspartame | 3.90 | 1.77 |
| 8 | Iron Oxide Red | 0.25 | 0.11 |
| 9 | Iron Oxide Yellow | 0.17 | 0.08 |
| 10 | Colloidal Silicon dioxide | 8.80 | 4.00 |
| 11 | Sodium Stearyl Fumarate | 11.0 | 5.00 |

| | | |
|---|---|---|
| Total | 220.00 | 100 |

A part of Crospovidone, Iron oxide red and Iron oxide yellow were sifted twice through #80 ASTM sieve. The resulting material was sifted along with Memantine Hydrochloride, a part of Colloidal Silicon dioxide, Mannitol, Lactose monohydrate, remaining portion of Crospovidone, Peppermint flavor, Acesulfame Potassium and Aspartame through #40 ASTM sieve. To this Sodium Stearyl Fumarate and remaining portion of Colloidal Silicon dioxide sifted together through #60 ASTM sieve was added and blended in a blender. The resulting blend was sifted through #40 ASTM sieve and after blending for required time compressed into tablets using suitable tooling.

### Disintegration:

The tablets were subjected to disintegration test as per United States Pharmacopoeia (USP) and the result is tabulated below.

TABLE 5

| Test | Disintegration Time (sec) |
|---|---|
| Disintegration Test as per USP | 14 |

### Dissolution:

The resulting tablets were also subjected to dissolution test using USP type I dissolution apparatus in 900 ml of dissolution medium (12 g sodium chloride in 6 liter water, pH adjusted to 1.2 with HCl) stirred at 100 rpm. The result is tabulated below.

TABLE 6

| Time (min) | Percentage Drug dissolved |
|---|---|
| 05 | 97.3 |
| 10 | 98.1 |
| 15 | 99.9 |
| 20 | 101.3 |

### Example 4

| S. No. | Ingredients | Quantity (mg/tab) | % W/W |
|---|---|---|---|
| 1 | Memantine Hydrochloride | 10.00 | 4.55 |
| 2 | Mannitol | 88.00 | 40.00 |
| 3 | Xylitol | 70.88 | 32.22 |
| 4 | Cross linked polyvinyl pyrrolidone | 16.50 | 7.50 |
| | (Crospovidone) | | |
| 5 | Peppermint Flavor | 3.90 | 1.77 |
| 6 | Acesulfame Potassium | 6.60 | 3.00 |
| 7 | Aspartame | 3.90 | 1.77 |
| 8 | Iron Oxide Red | 0.25 | 0.11 |
| 9 | Iron Oxide Yellow | 0.17 | 0.08 |
| 10 | Colloidal Silicon dioxide | 8.80 | 4.00 |
| 11 | Sodium Stearyl Fumarate | 11.0 | 5.00 |

| | | |
|---|---|---|
| Total | 220.00 | 100 |

A part of Crospovidone, Iron oxide red and Iron oxide yellow were sifted twice through #80 ASTM sieve. The resulting material was sifted along with Memantine Hydrochloride, a part of Colloidal Silicon dioxide, Mannitol, Xylitol, remaining portion of Crospovidone, Peppermint flavor, Acesulfame Potassium and Aspartame through #40 ASTM sieve. To this Sodium Stearyl Fumarate and remaining portion of Colloidal Silicon dioxide sifted together through #60 ASTM sieve was added and blended in a blender. The resulting blend was sifted through #40 ASTM sieve and after blending for required time compressed into tablets using suitable tooling.

### Disintegration:

The tablets were subjected to disintegration test as per United States Pharmacopoeia (USP) and the result is tabulated below.

TABLE 7

| Test | Disintegration Time (sec) |
|---|---|
| Disintegration Test as per USP | 19 |

### Dissolution:

The resulting tablets were also subjected to dissolution test using USP type I dissolution apparatus in 900 ml of dissolution medium (12 g sodium chloride in 6 liter water, pH adjusted to 1.2 with HCl) stirred at 100 rpm. The result is tabulated below.

TABLE 8

| Time (min) | Percentage Drug dissolved |
|---|---|
| 05 | 100.0 |
| 10 | 101.3 |
| 15 | 103.5 |
| 20 | 104.8 |

### Example 5

| S. | | | |
|---|---|---|---|
| No. | Ingredients | Quantity (mg/tab) | % W/W |
| 1 | Memantine Hydrochloride | 10.00 | 4.55 |
| 2 | Mannitol | 164.38 | 74.72 |
| 3 | Croscarmellose Sodium (Ac-Di-Sol) | 11.00 | 5.00 |
| 4 | Peppermint Flavor | 3.90 | 1.77 |
| 5 | Acesulfame Potassium | 6.60 | 3.00 |
| 6 | Aspartame | 3.90 | 1.77 |
| 7 | Iron Oxide Red | 0.25 | 0.11 |
| 8 | Iron Oxide Yellow | 0.17 | 0.08 |
| 9 | Colloidal Silicon dioxide | 8.80 | 4.00 |
| 10 | Sodium Stearyl Fumarate | 11.0 | 5.00 |

| | | |
|---|---|---|
| Total | 220.00 | 100 |

A part of AcDiSol, Iron oxide red and Iron oxide yellow were sifted twice through #80 ASTM sieve. The resulting material was sifted along with Memantine

Hydrochloride, a part of Colloidal Silicon dioxide, Mannitol, remaining portion of AcDiSol, Peppermint flavor, Acesulfame Potassium and Aspartame through #40 ASTM sieve. To this Sodium Stearyl Fumarate and remaining portion of Colloidal Silicon dioxide sifted together through #60 ASTM sieve was added and blended in a blender. The resulting blend was sifted through #40 ASTM sieve and after blending for required time compressed into tablets using suitable tooling.

### Disintegration:

The tablets were subjected to disintegration test as per United States Pharmacopoeia (USP) and the result is tabulated below.

TABLE 9

| Test | Disintegration Time (sec) |
|---|---|
| Disintegration Test as per USP | 19 |

### Dissolution:

The resulting tablets were also subjected to dissolution test using USP type I dissolution apparatus in 900 ml of dissolution medium (12 g sodium chloride in 6 liter water, pH adjusted to 1.2 with HCl) stirred at 100 rpm. The result is tabulated below.

TABLE 10

| Time (min) | Percentage Drug dissolved |
|---|---|
| 05 | 93.7 |
| 10 | 99.1 |
| 15 | 99.2 |
| 20 | 100.5 |

### Example 6

| S. | | | |
|---|---|---|---|
| No. | Ingredients | Quantity (mg/tab) | % W/W |
| 1 | Memantine Hydrochloride | 10.00 | 4.55 |
| 2 | Mannitol | 164.38 | 74.72 |
| 3 | Sodium Starch Glycolate (Primojel) | 11.00 | 5.00 |
| 4 | Peppermint Flavor | 3.90 | 1.77 |
| 5 | Acesulfame Potassium | 6.60 | 3.00 |
| 6 | Aspartame | 3.90 | 1.77 |
| 7 | Iron Oxide Red | 0.25 | 0.11 |
| 8 | Iron Oxide Yellow | 0.17 | 0.08 |
| 9 | Colloidal Silicon dioxide | 8.80 | 4.00 |
| 10 | Sodium Stearyl Fumarate | 11.0 | 5.00 |

| | | |
|---|---|---|
| Total | 220.00 | 100 |

A part of Primojel, Iron oxide red and Iron oxide yellow were sifted twice through #80 ASTM sieve. The resulting material was sifted along with Memantine Hydrochloride, a part of Colloidal Silicon dioxide, Mannitol, remaining portion of Primojel, Peppermint flavor, Acesulfame Potassium and Aspartame through #40 ASTM sieve. To this Sodium Stearyl Fumarate and remaining portion of Colloidal Silicon dioxide sifted together through #60 ASTM sieve was added and blended in a blender. The resulting blend was sifted through #40 ASTM sieve and after blending for required time compressed into tablets using suitable tooling.

### Disintegration:

The tablets were subjected to disintegration test as per United States Pharmacopoeia (LISP) and the result is tabulated below.

TABLE 11

| Test | Disintegration Time (sec) |
|---|---|
| Disintegration Test as per USP | 24 |

### Dissolution:

The resulting tablets were also subjected to dissolution test using USP type I dissolution apparatus in 900 ml of dissolution medium (12 g sodium chloride in 6 liter water, pH adjusted to 1.2 with HCl) stirred at 100 rpm. The result is tabulated below.

TABLE 12

| Time (min) | Percentage Drug dissolved |
|---|---|
| 05 | 92.4 |
| 10 | 93.3 |
| 15 | 93.9 |
| 20 | 96.2 |

### Example 7

| S. No. | Ingredients | Quantity (mg/tab) | % W/W |
|---|---|---|---|
| 1 | Memantine Hydrochloride | 10.00 | 4.55 |
| 2 | Mannitol | 120.38 | 54.72 |
| 3 | Cross linked polyvinyl pyrrolidone | 55.00 | 25.00 |
| | (Crospovidone) | | |
| 4 | Peppermint Flavor | 3.90 | 1.77 |
| 5 | Acesulfame Potassium | 6.60 | 3.00 |
| 6 | Aspartame | 3.90 | 1.77 |
| 7 | Iron Oxide Red | 0.25 | 0.11 |
| 8 | Iron Oxide Yellow | 0.17 | 0.08 |
| 9 | Colloidal Silicon dioxide | 8.80 | 4.00 |
| 10 | Sodium Stearyl Fumarate | 11.0 | 5.00 |

| | | |
|---|---|---|
| Total | 220.00 | 100 |

A part of Crospovidone, Iron oxide red and Iron oxide yellow were sifted twice through #80 ASTM sieve. The resulting material was sifted along with Memantine Hydrochloride, a part of Colloidal Silicon dioxide, Mannitol, remaining portion of Crospovidone, Peppermint flavor, Acesulfame Potassium and Aspartame through #40 ASTM sieve. To this Sodium Stearyl Fumarate and remaining portion of Colloidal Silicon dioxide sifted together through #60 ASTM sieve was added and blended in a blender. The resulting blend was sifted through #40 ASTM sieve and after blending for required time compressed into tablets, using suitable tooling.

### Disintegration:

The tablets were subjected to disintegration test as per United States Pharmacopoeia (USP) and the result is tabulated below.

TABLE 13

| Test | Disintegration Time (sec) |
|---|---|
| Disintegration Test as per USP | 18 |

### Dissolution:

The resulting tablets were also subjected to dissolution test using USP type I dissolution apparatus in 900 ml of dissolution medium (12 g sodium chloride in 6 liter water, pH adjusted to 1.2 with HCl) stirred at 100 rpm. The result is tabulated below.

TABLE 14

| Time (min) | Percentage Drug dissolved |
|---|---|
| 05 | 97.1 |
| 10 | 100.2 |
| 15 | 101.0 |
| 20 | 101.9 |

We claim:

1. A quick dissolve pharmaceutical composition comprising effective amount of memantine hydrochloride, wherein the composition disintegrates in less than 60 seconds.

2. The quick dissolve pharmaceutical composition according to claim 1, wherein the composition disintegrates in less than 30 seconds.

3. The quick dissolve pharmaceutical composition according to claim 1, wherein the said composition contains at least one water-soluble diluent and at least one disintegrant and optionally other pharmaceutically acceptable excipients.

4. The quick dissolve pharmaceutical composition according to claim 3 comprises the said water-soluble diluent in an amount from about 0.5% to about 90% of by weight of the composition.

5. The quick dissolve pharmaceutical composition according to claim 4, wherein the water-soluble diluent is one or more of mono and di saccharides selected from mannitol, sorbitol, xylitol, maltitol, sucrose, fructose and lactose.

6. The quick dissolve pharmaceutical composition according to claim 5, wherein the water-soluble diluent is mannitol.

7. The quick dissolve pharmaceutical composition according to claim 3 comprises the said disintegrant in an amount from, about 0.5% to about 50% of by weight of the composition.

8. The quick dissolve pharmaceutical formulation according to claim 7, wherein the disintegrant is one or, more of crospovidone, croscarmellose sodium, starch, starch derivatives or cellulose polymers.

9. The quick dissolve pharmaceutical composition according to claim 8, wherein the disintegrant is crospovidone.

10. The quick dissolve pharmaceutical composition according to claim 3, wherein the said pharmaceutically acceptable excipients is one or more of sweetening agent, flavoring agent, pH modifier, coloring agent, glidant, lubricant, anti-tacking agent and binder.

11. The quick dissolve pharmaceutical composition according to claim 1 is in the form of a tablet, pellets, granules or powder.

12. A process for preparing quick dissolve composition containing memantine hydrochloride comprising the steps of a) forming a mixture of memantine hydrochloride with one or more pharmaceutical excipients b) processing the mixture into an oral dosage form.

13. The process according to claim 12, wherein said pharmaceutical excipients includes at least one water soluble diluent and at least one disintegrant and optionally other excipienth selected from one or more of sweetening agent, flavoring agent, pH modifier, coloring agent, glidant, lubricant, anti-tacking agent and binder.

14. The process according to claim 13, wherein said water soluble diluent is one or more of mono and di saccharides selected from mannitol, sorbitol, xylitol, maltitol, sucrose, fructose and lactose.

15. The process according to claim 13, wherein said disintegrant is one or more of crospovidone, croscarmellose sodium, starch, starch derivatives or cellulose polymers.

16. The process according to claim 12, Wherein the said process of forming a mixture involves wet granulation, dry granulation or direct compression.

17. The process according to claim 12, wherein the said quick dissolve composition is in the form of a tablet, pellets, granules or powder.
